(19) United States
(12) Patent Application Publication
(10) Pub. No.: US 2006/0252825 A1
(43) Pub. Date: Nov. 9, 2006

(54) Crystal forms of {[(2r)-7-(2,6-dichlorophenyl)-5-fluoro-2,3-dihydro-1-benzofuran-2-yl]methyl}amine hydrochloride

(75) Inventors: Abdolsamad Tadayon, Kirkland (CA); Qing Yu, Laval (CA); Michel Bernatchez, Montreal (CA)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/409,303

(22) Filed: Apr. 21, 2006

Related U.S. Application Data
(60) Provisional application No. 60/674,318, filed on Apr. 22, 2005.

(51) Int. Cl. A61K 31/343; C07D 307/81

(52) U.S. Cl. 514/469; 549/467

(57) ABSTRACT

The present invention is directed to crystalline forms of the 5-HT_{2C} agonist {[(2R)-7-(2,6-dichlorophenyl)-5-fluoro-2,3-dihydro-1-benzofuran-2-yl]methyl}amine hydrochloride, as well as compositions, processes of preparation, and uses thereof.

This application claims benefit of priority to U.S. provisional patent application Ser. No. 60/674,318 filed on Apr. 22, 2005, which is hereby incorporated in its entirety.

### FIELD OF THE INVENTION

The present invention is directed to crystalline forms of the 5-HT_{2C} agonist {[(2R)-7-(2,6-dichlorophenyl)-5-fluoro-2,3-dihydro-1-benzofuran-2-yl]methyl}amine hydrochloride, as well as compositions, processes of preparation, and uses thereof.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, vol. 156, pp 1686-1696 (1999); Masand, P. S., Exp. Opin. Pharmacother. I: pp 377-389, (2000); Whitaker, R., Spectrum Life Sciences. Decision Resources. vol. 2, pp 1-9 (2000)).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology vol. 124, pp 57-73, (1996); Cowen, P. J., et. al., Human Psychopharmacology vol. 10, pp 385-391 (1995); Rosenzweig-Lipson, S., et. al., ASPET abstract (2000)).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology vol. 37, pp 265-272 (1998); Fox, S. H., et. al., Experimental Neurology vol. 151, pp 35-49 (1998)). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology vol. 37, pp 953-955 (1998); Di Matteo, V., et. al., Neuropharmacology vol. 38, pp 1195-1205 (1999); Di Giovanni, G., et. al., Synapse vol. 35, pp 53-61 (2000)), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

Certain dihydrobenzofurans are believed to be selective, potent agonists of the 5-HT_{2C} receptor and are therefore useful in a variety of applications, such as those recited above. The compound {[(2R)-7-(2,6-dichlorophenyl)-5-fluoro-2,3-dihydro-1-benzofuran-2-yl]methyl}amine, shown below in Formula I, is an example of a dihydrobenzofuran having such desirable characteristics. Preparation and characterization of this compound and its hydrochloric acid salt form (i.e. hydrochloride salt form) are described in U.S. Ser. Nos. 60/621,024 and 10/970,714, each of which is incorporated herein by reference in its entirety.

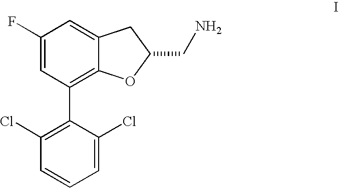

### {[(2R)-7-(2,6-dichlorophenyl)-5-fluoro-2,3-dihydro-1-benzofuran-2-yl]methyl}amine

Because improved drug formulations showing, for example, better bioavailability or better stability are consistently sought, there is an ongoing need for new or purer polymorphic forms of existing drug molecules. The crystalline polymorphs of {[(2R)-7-(2,6-dichlorophenyl)-5-fluoro-2,3-dihydro-1-benzofuran-2-yl]methyl}amine hydrochloride described herein are directed toward this end.

### SUMMARY OF THE INVENTION

The present invention provides crystalline polymorphs I, II, and III of {[(2R)-7-(2,6-dichlorophenyl)-5-fluoro-2,3-dihydro-1-benzofuran-2-yl]methyl}amine hydrochloride.

The present invention provides a crystalline polymorph (Form I) of {[(2R)-7-(2,6-dichlorophenyl)-5-fluoro-2,3-dihydro-1-benzofuran-2-yl]methyl}amine hydrochloride characterized by the XRPD and other data provided herein.

The present invention provides a crystalline polymorph (Form II) of {[(2R)-7-(2,6-dichlorophenyl)-5-fluoro-2,3-dihydro-1-benzofuran-2-yl]methyl}amine hydrochloride characterized by the XRPD and other data provided herein.

The present invention provides a crystalline polymorph (Form III) of {[(2R)-7-(2,6-dichlorophenyl)-5-fluoro-2,3-dihydro-1-benzofuran-2-yl]methyl}amine hydrochloride characterized by the XRPD and other data provided herein.

The present invention further provides compositions comprising at least one polymorph of the invention.

The present invention further provide processes of preparing the polymorphs of the invention comprising precipitating the polymorphs from a solution comprising {[(2R)-7-(2,6-dichlorophenyl)-5-fluoro-2,3-dihydro-1-benzofuran-2-yl]methyl}amine hydrochloride and a crystallizing solvent.

The present invention further provides polymorphs prepared by the processes of preparation described herein.

The present invention further provides methods of treating 5-HT_{2C} associated diseases and conditions such as those recited herein.

The present invention further provides use of a polymorph of the invention in therapy.

The present invention further provides use of a polymorph of the invention for the preparation of a medicament for use in therapy.

### BRIEF DESCRIPTION OF THE DRAWINGS

FIG. 1 depicts an X-ray powder diffraction (XRPD) pattern characteristic of Form I.

FIG. 2 depicts an X-ray powder diffraction (XRPD) pattern characteristic of Form II.

FIG. 3 depicts an X-ray powder diffraction (XRPD) pattern characteristic of transient Form III.

FIG. 4 depicts a differential scanning calorimetry (DSC) thermogram characteristic of Form I.

FIG. 5 depicts a differential scanning calorimetry (DSC) thermogram characteristic of Form II.

### DETAILED DESCRIPTION

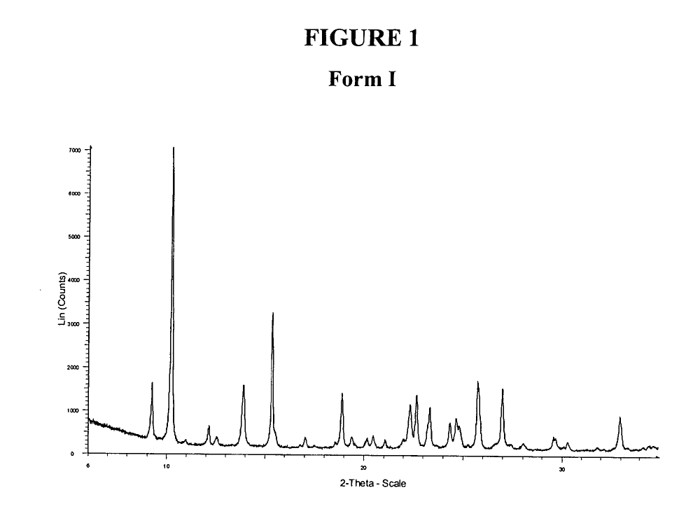

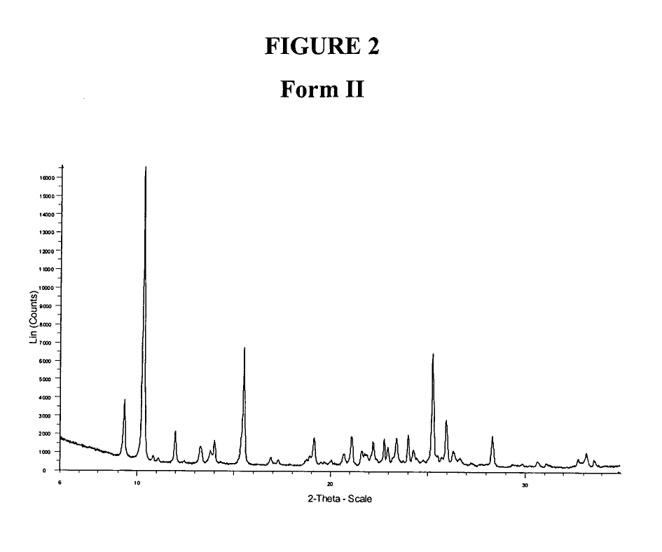

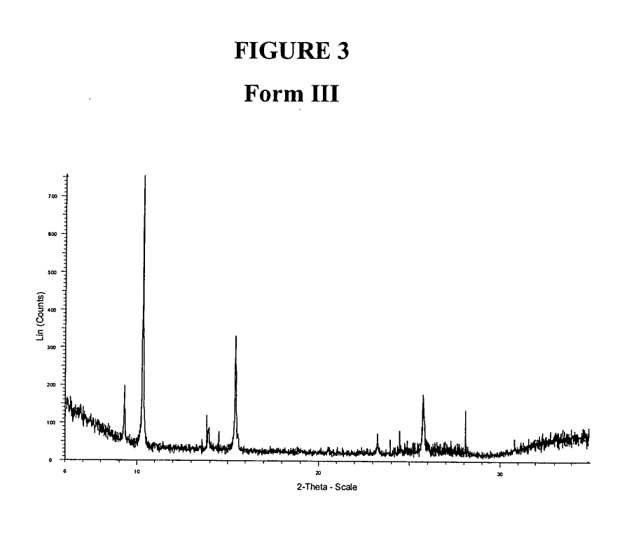

The present invention provides, inter alia, an anhydrous, non-solvated crystalline polymorph of the 5-HT_{2C} agonist {[(2R)-7-(2,6-dichlorophenyl)-5-fluoro-2,3-dihydro-1-benzofuran-2-yl]methyl}amine hydrochloride referred to herein as Form I. The present invention further provides an anhydrous, non-solvated crystalline polymorph of {[(2R)-7-(2,6-dichlorophenyl)-5-fluoro-2,3-dihydro-1-benzofuran-2-yl]methyl}amine hydrochloride referred to herein as Form II. Each of the polymorphs can be identified by one or more solid state analytical methods such as X-ray powder diffraction. For example, Form I can be identified by its powder X-ray diffraction pattern which is provided in FIG. 1 and Form II can be identified by its powder X-ray diffraction pattern which is provided in FIG. 2. Powder X-ray diffraction data consistent with Forms I and II are provided in Tables 1 and 2 below. FIG. 3 and Table 3 further provide X-ray powder diffraction data characteristic of another crystalline form of {[(2R)-7-(2,6-dichlorophenyl)-5-fluoro-2,3-dihydro-1-benzofuran-2-yl]methyl}amine hydrochloride referred to herein as Form II. Collection parameters for the X-ray data provided herein were as follows: 6.00-35.00 degree range, using a Bruker D8 Advance machine, with no Ni filter.

TABLE 1
(Form I)

| Degree (2θ) | Intensity (%) |
|---|---|
| 9.2 | 32.7 |
| 10.2 | 100.0 |
| 12.1 | 20.3 |
| 12.5 | 17.3 |
| 13.9 | 32.2 |
| 15.4 | 53.0 |
| 17.0 | 17.5 |
| 18.9 | 29.9 |
| 19.4 | 17.6 |
| 20.1 | 17.0 |
| 20.5 | 17.8 |
| 21.0 | 16.8 |
| 22.3 | 26.9 |
| 22.7 | 29.6 |
| 23.3 | 25.9 |
| 24.4 | 21.6 |
| 24.7 | 22.8 |
| 25.8 | 33.1 |
| 27.0 | 31.5 |
| 28.0 | 16.0 |
| 29.7 | 17.1 |
| 30.3 | 16.4 |
| 33.0 | 23.9 |

TABLE 2
(Form II)

| Degree (2θ) | Intensity (%) |
|---|---|
| 9.3 | 23.1 |
| 10.3 | 100.0 |
| 12.0 | 13.1 |
| 12.4 | 2.9 |
| 13.3 | 8.1 |
| 13.8 | 6.4 |
| 14.0 | 10.1 |
| 15.5 | 42.0 |
| 16.9 | 4.3 |
| 17.3 | 3.6 |
| 19.1 | 10.8 |
| 20.7 | 5.7 |
| 21.1 | 11.5 |
| 21.7 | 6.7 |
| 21.8 | 5.6 |
| 22.2 | 9.4 |
| 22.8 | 10.8 |
| 23.0 | 7.7 |
| 23.4 | 11.1 |
| 24.0 | 12.1 |
| 24.3 | 7.1 |
| 25.3 | 40.3 |
| 26.0 | 17.2 |
| 26.4 | 6.7 |
| 26.7 | 4.4 |
| 28.4 | 11.9 |
| 30.7 | 3.4 |
| 31.1 | 2.6 |
| 32.8 | 4.4 |
| 33.2 | 6.5 |
| 33.6 | 3.8 |

TABLE 3
(Form III)

| Degree (2θ) | Intensity (%) |
|---|---|
| 9.3 | 25.9 |
| 10.3 | 100.0 |
| 13.9 | 10.8 |
| 15.4 | 43.7 |
| 23.3 | 9.4 |
| 25.8 | 22.3 |

The relative intensities of the peaks on XRD can vary depending on, inter alia, the sample preparation technique, crystal size distribution, the sample mounting procedure, and the particular instrument employed. Moreover, instrument variation and other factors can affect the 2-theta values. Therefore, the term “substantially” in the context of XRPD is meant to encompass that peak assignments can vary by plus or minus about 0.2°. Moreover, new peaks may be observed or existing peaks may disappear, depending on the type of the machine or the settings (for example, whether a Ni filter is used or not on a Bruker D8 Advance machine).

In some embodiments, Form I has a powder X-ray diffraction pattern comprising a characteristic peak, in terms of 2θ, at about 10.2° and at least one characteristic peak, in terms of 2θ, selected from about 27.0° and about 25.8°. In further embodiments the powder X-ray diffraction pattern comprises characteristic peaks, in terms of 2θ, at about 10.2°, about 25.8°, and about 27.0°. In yet further embodiments, the powder X-ray diffraction pattern further comprises a characteristic peak, in terms of 2θ, at about 12.1°. In some embodiments, the powder X-ray diffraction pattern comprises at least four characteristic peaks, in terms of 2θ, selected from about 9.2°, about 10.2°, about 12.1°, about 13.9°, about 15.4°, about 18.9°, about 22.3°, about 22.7°, about 23.3°, about 25.8°, about 27.0°, and about 33.0°. In yet further embodiments, Form I is characterized by a powder X-ray diffraction pattern substantially as shown in FIG. 1.

In some embodiments, Form II has a powder X-ray diffraction pattern comprising a characteristic peak, in terms of 2°, at about 10.3° and at least one characteristic peak, in terms of 2θ, selected from about 25.3°, about 26.0°, and about 28.4°. In further embodiments, the powder X-ray diffraction pattern comprises characteristic peaks, in terms of 2θ, at about 10.3°, about 25.3°, and about 26.0°. In yet further embodiments, the powder X-ray diffraction pattern further comprises a characteristic peak, in terms of 2θ, at about 11.9°. In some embodiments, the powder X-ray diffraction pattern comprises at least four characteristic peaks, in terms of 2θ, selected from about 9.3°, about 10.3°, about 11.9°, about 12.4°, about 15.5°, about 19.1°, about 21.1°, about 22.3°, about 22.8°, about 23.4°, about 24.0°, about 25.3°, about 26.0°, and about 28.4°. In yet further embodiments, Form II is characterized by a powder X-ray diffraction pattern substantially as shown in FIG. 2.

In some embodiments, Form II is characterized by a crystalline habit which is substantially needle-shaped.

In some embodiments, Form III has a powder X-ray diffraction pattern comprising characteristic peaks, in terms of 2θ, at about 10.3° and about 15.4° and having an absence of peaks from about 17.0° to about 22.0°. In further embodiments, the diffraction pattern has an absence of peaks from about 18.0° to about 21.0°. In some embodiments, the diffraction pattern has an absence of peaks from about 17.0° to about 20.0°. In some further embodiments, the diffraction pattern has an absence of peaks from about 18.0° to about 20.0°. In yet further embodiments, the diffraction pattern further comprises a characteristic peak at about 25.8°. In some embodiments, Form III has an X-ray powder diffraction pattern substantially as shown in FIG. 3.

As used herein, the phrase “absence of peaks” is meant to refer to a region of the X-ray powder diffraction pattern with no peak having a relative intensity more than about 2%.

The polymorphic forms of the invention are readily distinguishable from other each other particularly by their physical properties. Sample data are compared for Forms I and II below in Table 4.

TABLE 4

| Measurement | Form I | Form II |
|---|---|---|
| Solubility-Water (mg/mL) | 67 | 51 |
| DSC | Single Melting | Single Melting |
| | Endotherm | Endotherm |
| | about 234° C. | about 234° C. |
| TGA (% weight loss) | 0.1-0.2 | — |
| Discriminatory X-Ray Powder | 25.8° and 27.0° | 25.3°, 26.0°, and 28.4° |
| peaks (2□) | | |

As can be seen in Table 4, the two crystalline polymorphs have discernable physical and spectroscopic characteristics. Based on solubility data, Form II appears to be thermodynamically more stable in water than Form I. Accordingly, the increased stability of Form II could facilitate manufacturing and purification processes. Form II would also be expected to have better resistance to degradation brought on by, for example, exposure to high temperatures and/or humidity, and have a longer shelf-life than Form I or amorphous material. In contrast, the higher solubility of Form I in water can be advantageous with respect to potential bioavailability.

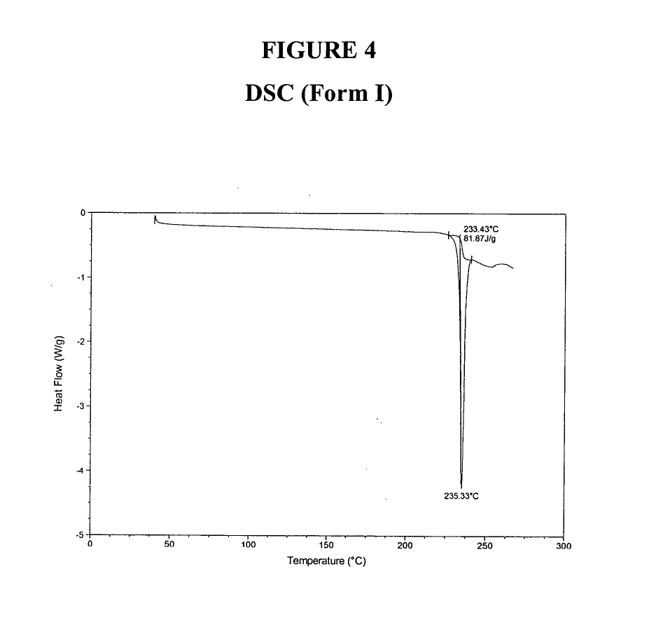

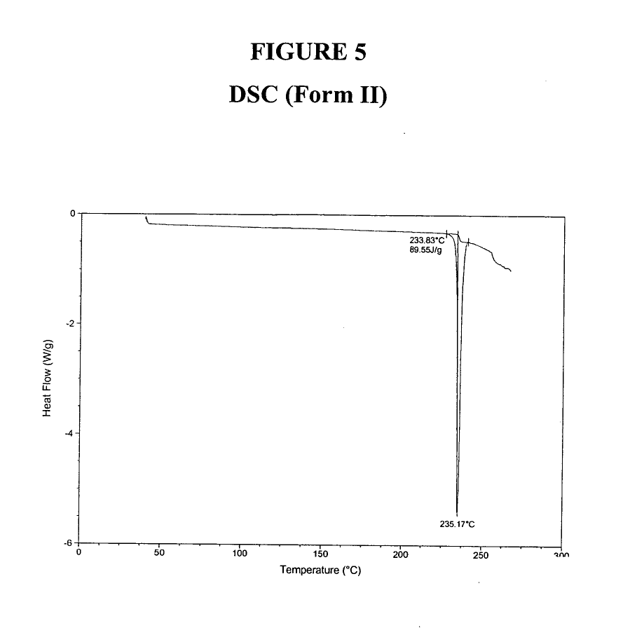

The DSC scans of Forms I and II are depicted in FIGS. 4 and 5. The melting points of Forms I and II are both about 234° C. (onset temperature with an apex at about 235° C.). The location of DSC peaks obtained for Forms I and II may shift depending on, inter alia, the particle size distribution, the heating rate, and the type of the machine. Accordingly, the temperature reading can vary about 4° C. DSC data were collected using a TA instrument model Q1000 with a heating rate of 10° C./min.

Crystalline Form I of the invention can be prepared according to routine methods in the art. For example, Form I can be precipitated from a solution of {[(2R)-7-(2,6-dichlorophenyl)-5-fluoro-2,3-dihydro-1-benzofuran-2-yl]methyl}amine hydrochloride in a crystallizing solvent. The crystallizing solvent can contain any suitable organic solvent. In some embodiments, the crystallizing solvent is a non-polar or weakly polar organic solvent. Example non-polar or weakly polar organic solvents include ethers and hydrocarbons. Example crystallizing solvents for precipitating Form I include ethers such as t-butylmethyl ether, diethyl ether, tetrahydrofuran, dimethoxymethane, 1,3-dioxane, 1,4-dioxane, furan, ethylene glycol dimethyl ether, ethylene glycol diethyl ether, diethylene glycol dimethyl ether, diethylene glycol diethyl ether, triethylene glycol dimethyl ether, anisole, and the like; hydrocarbons such as pentane, hexanes, heptanes, benzene, toluene, and the like; alcohols such as methanol, ethanol, 2-nitroethanol, 2-fluoroethanol, 2,2,2-trifluoroethanol, ethylene glycol, 1-propanol, isopropanol (2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, and the like; or other solvents such as ethyl acetate. In some embodiments, the crystallizing solvent is t-butyl methyl ether.

Suitable crystallizing solvents further include mixtures of the aforementioned solvents as well as mixtures of the aforementioned solvents with water (e.g., isopropanol/water).

In some embodiments, Form I is prepared by combining {[(2R)-7-(2,6-dichlorophenyl)-5-fluoro-2,3-dihydro-1-benzofuran-2-yl]methyl}amine (free base) with HCl in a suitable solvent containing an ether such as t-butylmethyl ether and precipitating Form I from the solution. In further embodiments, the solution of {[(2R)-7-(2,6-dichlorophenyl)-5-fluoro-2,3-dihydro-1-benzofuran-2-yl]methyl}amine hydrochloride is optionally seeded with Form I seed crystals of {[(2R)-7-(2,6-dichlorophenyl)-5-fluoro-2,3-dihydro-1-benzofuran-2-yl]methyl}amine hydrochloride.

Crystalline Form II of the invention can be prepared according to any of numerous methods routine in the art. For example, Form II can be precipitated from a solution of {[(2R)-7-(2,6-dichlorophenyl)-5-fluoro-2,3-dihydro-1-benzofuran-2-yl]methyl}amine hydrochloride in a crystallizing solvent. The crystallizing solvent can be any suitable solvent such as a polar organic solvent, water, or mixture thereof. Example polar organic solvents include alcohols, such as methanol, ethanol, 2-nitroethanol, 2-fluoroethanol, 2,2,2-trifluoroethanol, ethylene glycol, 1-propanol, isopropanol (2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, or glycerol, and the like; water or water/organic solvent mixtures; or other solvents such as acetone. Some example solvents include water/alcohol mixtures such as isopropanol or other alcohols containing about 1 to about 10%, about 1 to about 5%, or about 3 to about 4% by weight water. In some embodiments, Form II is prepared by combining {[(2R)-7-(2,6-dichlorophenyl)-5-fluoro-2,3-dihydro-1-benzofuran-2-yl]methyl}amine (free base) with HCl in a suitable solvent containing an alcohol such as isopropanol and precipitating Form II from the solution. In further embodiments, the solution of {[(2R)-7-(2,6-dichlorophenyl)-5-fluoro-2,3-dihydro-1-benzofuran-2-yl]methyl}amine hydrochloride is optionally seeded with Form II seed crystals of {[(2R)-7-(2,6-dichlorophenyl)-5-fluoro-2,3-dihydro-1-benzofuran-2-yl]methyl}amine hydrochloride.

Crystalline Form II can also be prepared by converting Form I to Form II using any of numerous routine methods. In some embodiments, Form I is wholly or partially converted to Form II by slurrying Form I in an appropriate organic solvent, water, or mixture thereof. In some embodiment, Form II is prepared by slurrying Form I in water or a solvent mixture containing water.

Crystalline Form III of the invention can be prepared according to any of numerous methods routine in the art. For example, Form III can be made by slurrying {[(2R)-7-(2,6-dichlorophenyl)-5-fluoro-2,3-dihydro-1-benzofuran-2-yl]methyl}amine hydrochloride having Form I in water. In some embodiments, Form I is slurried at a temperature of about 20 to about 30° C., such as about 25° C. In some embodiments, Form I is slurried in water for about 1-3 days.

Precipitation of the crystalline forms of the invention can be carried out by any suitable manner according to routine methods. For example, solutions of {[(2R)-7-(2,6-dichlorophenyl)-5-fluoro-2,3-dihydro-1-benzofuran-2-yl]methyl}amine hydrochloride can be evaporated, cooled, treated with antisolvent, or combinations thereof. Treatment with antisolvent can be carried out by layering or vapor diffusion techniques. Suitable antisolvents include organic solvents, as well as water, that are miscible with the crystallizing solvent, yet are relatively poor solvents for the subject compound. In some embodiments, precipitation is carried out where the solution of {[(2R)-7-(2,6-dichlorophenyl)-5-fluoro-2,3-dihydro-1-benzofuran-2-yl]methyl}amine hydrochloride is heated to a temperature of about 40 to about 100, about 50 to about 90, about 60 to about 80, or about 70 to about 80° C., typically until all solids are dissolved, and then cooled to a temperature below about 60, below about 50, below about 40, below about 30, below about 20, below about 10, or below about 0° C. In some embodiments, the solution is heated to a temperature of about 60 to about 80° C. and then cooled to a temperature below about 60° C.

Crystal forms of the invention can be further processed to modulate particle size. For example, the crystal forms of the invention can be milled to reduce average crystal size and/or to prepare a sample suitable for manipulation and formulation.

The present invention further provides compositions containing a polymorph of the invention. In some embodiments, at least about 50%, about 70%, about 80%, about 90%, about 95%, about 97%, about 98.0%, about 98.1%, about 98.2%, about 98.3%, about 98.4%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8%, or about 99.9% by weight of total {[(2R)-7-(2,6-dichlorophenyl)-5-fluoro-2,3-dihydro-1-benzofuran-2-yl]methyl}amine hydrochloride in a composition is present as Form I or Form II. In further embodiments, compositions of the present invention consist essentially of {[(2R)-7-(2,6-dichlorophenyl)-5-fluoro-2,3-dihydro-1-benzofuran-2-yl]methyl}amine hydrochloride where at least about 95%, about 97%, or about 98.0%, about 98.1%, about 98.2%, about 98.3%, about 98.4%, about 98.5%, about 98.6%, about 98.7%, about 98.8%, about 98.9%, about 99.0%, about 99.1%, about 99.2%, about 99.3%, about 99.4%, about 99.5%, about 99.6%, about 99.7%, about 99.8%, or about 99.9% by weight of the {[(2R)-7-(2,6-dichlorophenyl)-5-fluoro-2,3-dihydro-1-benzofuran-2-yl]methyl}amine hydrochloride is present in the composition as either Form I or Form II. In some embodiments, the remainder {[(2R)-7-(2,6-dichlorophenyl)-5-fluoro-2,3-dihydro-1-benzofuran-2-yl]methyl}amine hydrochloride is present as amorphous material or other crystalline form. In some embodiments, the composition contains a mixture of Forms I and II. Respective amounts of polymorphic forms of {[(2R)-7-(2,6-dichlorophenyl)-5-fluoro-2,3-dihydro-1-benzofuran-2-yl]methyl}amine hydrochloride in a composition can be determined by any suitable spectroscopic method, such as X-ray powder diffraction.

The polymorphs of the invention are useful as 5-HT_{2C} agonists in methods of treating, for example, schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease.

The polymorphs of the invention are further useful in methods for treating bipolar disorders, depressive disorders, mood episodes, anxiety disorders, adjustment disorders, or eating disorders. In some embodiments, the bipolar disorder is bipolar I disorder, bipolar II disorder, or cyclothymic disorder; the depressive disorder is major depressive disorder, dysthymic disorder, or substance-induced mood disorder; the mood episode is major depressive episode, manic episode, mixed episode, or hypomanic episode; the anxiety disorder is panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder.

The polymorphs of the invention are further useful in methods for treating pain, urinary incontinence, substance abuse, addiction to alcohol and other drugs including cocaine and nicotine, epilepsy, sleep disorders, migraines, sexual dysfunction, gastrointestinal disorders, or obesity.

The polymorphs of the invention are further useful in methods for treating central nervous system deficiency associated with trauma, stroke, or spinal cord injury.

Methods of treating the diseases listed herein are understood to involve administering to a patient in need of such treatment a therapeutically effective amount of the polymorph of the invention, or composition containing the same. As used herein, the term “treating” in reference to a disease is meant to refer to preventing, inhibiting and/or ameliorating the disease.

As used herein, the term “patient” refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

In certain embodiments, the invention relates to compositions comprising at least one polymorph of the invention, and one or more pharmaceutically acceptable carriers, excipients, or diluents. Such compositions are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

The polymorphs of the invention can be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The polymorphs of the invention can be administered rectally or vaginally in the form of a conventional suppository. For administration by intranasal or intrabronchial inhalation or insufflation, the polymorphs of the invention can be formulated into an aqueous or partially aqueous solution, which can then be utilized in the form of an aerosol. The polymorphs of the invention can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The amount of polymorph provided to a patient will vary depending upon what is being administered, the purpose of the administration, such as prophylaxis or therapy, the state of the patient, the manner of administration, and the like. In therapeutic applications, polymorphs of the invention are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case can be subjectively determined by the attending physician. The variables involved include the specific condition and the size, age, and response pattern of the patient. Generally, a starting dose is about 5 mg per day with gradual increase in the daily dose to about 150 mg per day, to provide the desired dosage level in the patient.

In order that the invention disclosed herein may be more efficiently understood, examples are provided below. It should be understood that these examples are for illustrative purposes only and are not to be construed as limiting the invention in any manner.

### EXAMPLES

### Example 1

Preparation of Form I (No Seeding)

To 100 mL of t-butyl methyl ether were added 10 g of {[(2R)-7-(2,6-dichlorophenyl)-5-fluoro-2,3-dihydro-1-benzofuran-2-yl]methyl}amine (free base) and the resulting mixture was heated to 52° C. Then 2.7 mL HCl solution (36% wt/wt) was added and the temperature was decreased to room temperature over the course of 2 hours. A crystalline solid precipitated which corresponded to Form I according to XRPD. Yield about 90%.

### Example 2

Conversion of Form I to Form II

Form I was slurried in water for 1 day and for 5 days at 25° C. with stirring. After 1 day, XRPD revealed substantial conversion of the solid from Form I to Form II. After 5 days, no detectable amount of Form I was observed.

### Example 3

Preparation of Form II with Seeding

Method A

To 15 mL of isopropyl alcohol was added 3.1 g of {[(2R)-7-(2,6-dichlorophenyl)-5-fluoro-2,3-dihydro-1-benzofuran-2-yl]methyl}amine (free base). The mixture was heated to 75° C. and the resulting suspension was stirred (RPM=200) until all solids dissolved. Then 1 g of HCl solution (36% wt/wt) was added to the base solution over the course of 1 minute. No nucleation was observed. Seeds of Form II were added and the suspension was stirred for 30 min. The temperature was decreased to 0° C. over the course of 4.5 hours. Crystalline solid having Form II was recovered (about 80% yield).

Method B

To 642 g of isopropyl alcohol (IPA) were added 260 g of free base at room temperature. To the solution at 15-25° C. was added 218 g of a solution of HCl:IPA (16.7% HCl by weight; about 1.2 eq HCl relative to free base). To the resulting white suspension was added 196 g IPA. The total IPA added was 5 volumes based on weight of free base. To the suspension was added 49.4 mL water. The resulting mixture was heated to 75-78° C. and stirred until the solids dissolved. The solution was then cooled to 65° C. over the course of 30 min, twice seeding using Form II crystals, once at 70° C. and another time at 65° C. The mixture was stirred at 65° C. for 30 min and then cooled to 55° C. over the course of 1 hour and then stirred at 55° C. for 1 hour. The white suspension was then cooled to 30-33° C. over the course of 1 h. The suspension was concentrated by reduced pressure distillation to 60% of the original volume. To the concentrate was added 5 volumes of IPA and the suspension was concentrated again under reduced pressure to 60% of the original volume. The suspension was then cooled to −10° C. over 1 h and stirred at −10° C. for 1 hr. The suspension was filtered and dried at 55° C. under vacuum providing crystalline product characterized as Form II. Yield about 85%.

### Example 4

Solubility of Forms I and II

Solubility was measured using the gravimetric method by separately suspending Form I and Form II in water with stirring at room temperature for 6 hours. Solubility of Form I was determined to be 67 mg/mL and solubility of Form II was determined to be 51 mg/mL (a repeat experiment of Form II resulted in 50 mg/mL). Because the solubility of Form II was less than Form I, it is believed that Form II is thermodynamically more stable than Form I.

### Example 5

Assessment of Stability in Water

Form I, Form II or a mixture thereof was slurried in water at 25° C. to assess stability. Results are provided in Tables A, B, C and D below. Generally, Form I converted to Form II under most of the conditions tested. Form II appeared to be stable in water. In one instance, Form I failed to convert to From II within the time frame tested, and in another instance, Form I converted to a putative Form III before converting to Form II (See FIG. 3). XRPD was used to identify crystalline form.

TABLE A

| Original Form | Form after 2 days | Form after 4 days |
|---|---|---|
| I | I, trace II | Increased amount of II |
| 50:50 I:II | trace I | — |
| II | II | II |
| I seeded with II | increased amount of II | increased amount of II |
| II seeded with I | II | II |

TABLE B

| Original Form | Form after 1 day | Form after 8 days |
|---|---|---|
| I | partial II | II |
| I | trace II | II, trace I |
| I | III | II |

TABLE C

| Original Form | Form after 3 days | Form after 9 days |
|---|---|---|
| I | mainly II, some I | II |
| I | I | — |
| I | I | I |

TABLE D

| Original Form | Form after 2 days | Form after 5 days |
|---|---|---|
| II | II | II |
| II | II | II |

### Example 6

Assessment of Stability in Organic Solvents

Form I was slurried in a variety of organic solvents at 50° C. for 30 hours. Results are provided in Table E below. In sum, Form I converted to Form II in acetone in ethanol, and remained stable in the other solvents tested. XRPD confirmed crystalline form.

TABLE E

| Solvent | Final Form |
|---|---|
| ethyl acetate | I |
| acetone | II |
| ethanol | II |
| toluene | I |
| heptane | I |
| t-butylmethyl ether | I |

### Example 7

Conversion of Form II to Form I

Form II was slurried in t-butylmethyl ether for 4 days at room temperature. The resulting solid was characterized by XRPD as Form I.

### Example 8

Effects of Seeding and Stirring Speed on Preparation of Forms I and II

The influence of seeding and/or stirring speed on the formation of different crystalline forms was tested. For each trial, 2.77 g of {[(2R)-7-(2,6-dichlorophenyl)-5-fluoro-2,3-dihydro-1-benzofuran-2-yl]methyl}amine hydrochloride was dissolved in 20 mL of isopropyl alcohol containing 2.5 wt % water at 75° C. The resulting solution was cooled to 60° C. over the course of 10 minutes. At this point, the solution was seeded with Form I, Form II, or were not seeded. Stirrer speed was set to 160 or 700 rpm. The solution was cooled to 0° C. and stirred for 12 hours. Crystalline form was detected by XRPD. Results are provided in Table F.

TABLE F

| Seeded with | Stir Rate (rpm) | Final Form | Yield |
|---|---|---|---|
| II | 160 | II | 80% |
| II | 700 | II | 80% |
| none | 160 | I | 77% |
| none | 700 | I | 70% |
| I | 160 | I | — |
| I | 700 | I | — |

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A crystalline polymorph (Form I) of {[(2R)-7-(2,6-dichlorophenyl)-5-fluoro-2,3-dihydro-1-benzofuran-2-yl]methyl}amine hydrochloride having a powder X-ray diffraction pattern comprising a characteristic peak, in terms of 2θ, at about 10.2° and at least one characteristic peak, in terms of 2θ, selected from about 27.0° and about 25.8°.

2. The polymorph of claim 1 wherein said powder X-ray diffraction pattern comprises at least four characteristic peaks, in terms of 2θ, selected from about 9.2°, about 10.2°, about 12.1°, about 13.9°, about 15.4°, about 18.9°, about 22.3°, about 22.7°, about 23.3°, about 25.8°, about 27.0°, and about 33.0°.

3. The polymorph of claim 1 having a powder X-ray diffraction pattern substantially as shown in FIG. 1.

4. The polymorph of claim 1 having a differential scanning calorimetry endotherm at about 234° C.

5. A composition comprising the polymorph of claim 1.

6. The composition of claim 5 wherein at least about 90% by weight of total {[(2R)-7-(2,6-dichlorophenyl)-5-fluoro-2,3-dihydro-1-benzofuran-2-yl]methyl}amine hydrochloride in said composition is present as said polymorph.

7. A composition comprising the polymorph of claim 1 and a pharmaceutically acceptable carrier.

8. A composition consisting essentially of the compound {[(2R)-7-(2,6-dichlorophenyl)-5-fluoro-2,3-dihydro-1-benzofuran-2-yl]methyl}amine hydrochloride wherein at least 95% by weight of said compound is present in said composition as the polymorph of claim 1.

9. A process for preparing the polymorph of claim 1 comprising precipitating said polymorph from a solution comprising {[(2R)-7-(2,6-dichlorophenyl)-5-fluoro-2,3-dihydro-1-benzofuran-2-yl]methyl}amine hydrochloride and a crystallizing solvent.

10. The process of claim 9 wherein said crystallizing solvent comprises t-butylmethyl ether.

11. A polymorph prepared by the process of claim 9.

12. A crystalline polymorph (Form II) of {[(2R)-7-(2,6-dichlorophenyl)-5-fluoro-2,3-dihydro-1-benzofuran-2-yl]methyl}amine hydrochloride having a powder X-ray diffraction pattern comprising a characteristic peak, in terms of 2θ, at about 10.3° and at least one characteristic peak, in terms of 2θ, selected from about 25.3°, about 26.0°, and about 28.4°.

13. The polymorph of claim 12 wherein said powder X-ray diffraction pattern comprises characteristic peaks, in terms of 2θ, at about 10.3°, about 25.3°, and about 26.0°.

14. The polymorph of claim 12 wherein said powder X-ray diffraction pattern comprises at least four characteristic peaks, in terms of 2θ, selected from about 9.3°, about 10.3°, about 11.9°, about 12.4°, about 15.5°, about 19.1°, about 21.1°, about 22.3°, about 22.8°, about 23.4°, about 24.0°, about 25.3°, about 26.0°, and about 28.4°.

15. The polymorph of claim 12 having a powder X-ray diffraction pattern substantially as shown in FIG. 2.

16. The polymorph of claim 12 having a differential scanning calorimetry endotherm at about 234° C.

17. The polymorph of claim 12 having a crystal habit which is substantially needle-shaped.

18. A composition comprising the polymorph of claim 12.

19. The composition of claim 18 wherein at least about 50% by weight of total {[(2R)-7-(2,6-dichlorophenyl)-5-fluoro-2,3-dihydro-1-benzofuran-2-yl]methyl}amine hydrochloride in said composition is present as said polymorph.

20. The composition of claim 18 wherein at least about 90% by weight of total {[(2R)-7-(2,6-dichlorophenyl)-5-fluoro-2,3-dihydro-1-benzofuran-2-yl]methyl}amine hydrochloride in said composition is present as said polymorph.

21. The composition of claim 18 wherein at least about 99.0% by weight of total {[(2R)-7-(2,6-dichlorophenyl)-5-fluoro-2,3-dihydro-1-benzofuran-2-yl]methyl}amine hydrochloride in said composition is present as said polymorph.

22. A composition comprising the polymorph of claim 12 and a pharmaceutically acceptable carrier.

23. A composition consisting essentially of the compound {[(2R)-7-(2,6-dichlorophenyl)-5-fluoro-2,3-dihydro-1-benzofuran-2-yl]methyl}amine hydrochloride wherein at least 95% by weight of said compound is present in said composition as the polymorph of claim 12.

24. A composition consisting essentially of the compound {[(2R)-7-(2,6-dichlorophenyl)-5-fluoro-2,3-dihydro-1-benzofuran-2-yl]methyl}amine hydrochloride wherein at least 99.0% by weight of said compound is present in said composition as the polymorph of claim 12.

25. A process for preparing the polymorph of claim 12 comprising precipitating said polymorph from a solution comprising {[(2R)-7-(2,6-dichlorophenyl)-5-fluoro-2,3-dihydro-1-benzofuran-2-yl]methyl}amine hydrochloride and a crystallizing solvent.

26. The process of claim 25 wherein said crystallizing solvent comprises an alcohol.

27. The process of claim 26 wherein said alcohol is isopropanol.

28. The process of claim 25 further comprising seeding said solution with seed crystals of Form II of {[(2R)-7-(2,6-dichlorophenyl)-5-fluoro-2,3-dihydro-1-benzofuran-2-yl]methyl}amine hydrochloride.

29. The process of claim 25 wherein said precipitating is carried out by cooling said solution.

30. A polymorph prepared by the process of claim 25.

31. A crystalline polymorph (Form II) of {[(2R)-7-(2,6-dichlorophenyl)-5-fluoro-2,3-dihydro-1-benzofuran-2-yl]methyl}amine hydrochloride having a powder X-ray diffraction pattern comprising characteristic peaks, in terms of 2θ, at about 10.3° and about 14.4° and having an absence of peaks from about 17.0° to about 22.0°.

32. The polymorph of claim 31 having an absence of peaks from about 18.0° to about 21.0°.

33. The polymorph of claim 31 having an X-ray powder diffraction pattern substantially as shown in FIG. 3.

34. A composition comprising the polymorph of claim 31.

35. A process for preparing the polymorph of claim 31 comprising slurrying crystalline {[(2R)-7-(2,6-dichlorophenyl)-5-fluoro-2,3-dihydro-1-benzofuran-2-yl]methyl}amine hydrochloride having Form I in water.

36. A method for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease comprising administering to a patient a therapeutically effective amount of a polymorph of claim 12.

37. The method of claim 36 wherein said patient is suffering from schizophrenia.

38. A method for treating bipolar disorders, depressive disorders, mood episodes, anxiety disorders, adjustment disorders, or eating disorders comprising administering to a patient a therapeutically effective amount of a polymorph of claim 12.

39. The method of claim 38 wherein the bipolar disorder is bipolar I disorder, bipolar II disorder, or cyclothymic disorder; the depressive disorder is major depressive disorder, dysthymic disorder, or substance-induced mood disorder; the mood episode is major depressive episode, manic episode, mixed episode, or hypomanic episode; the anxiety disorder is panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder.

40. A method for treating pain, urinary incontinence, substance abuse, addiction to alcohol and other drugs, epilepsy, sleep disorders, migraines, sexual dysfunction, gastrointestinal disorders, or obesity comprising administering to a patient a therapeutically effective amount of the polymorph of claim 12.
